(19) United States
(12) Patent Application Publication
(10) Pub. No.: US 2009/0093476 A1
(43) Pub. Date: Apr. 9, 2009

(54) PYRROLO[1,2-A]QUINOXALINE DERIVATIVES AS ADENOSINE A3 RECEPTOR MODULATORS AND USES THEREOF

(75) Inventors: Stephan Schann, Geispolsheim (FR); Stanislas Mayer, Eschau (FR); Sophie Gardan, St Denis en Val (FR)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: FAUST PHARMACEUTICALS, Illkirch (FR)

(21) Appl. No.: 12/158,035

(22) PCT Filed: Dec. 19, 2006
(86) PCT No.: PCT/EP2006/012258

§ 371 Date: Oct. 31, 2008

(30) Foreign Application Priority Data
Dec. 19, 2005 (EP) 05292739.9

(51) Int. Cl. A61K 31/519; C07D 487/04; A61P 1/00; A61P 37/00; A61K 31/5377

(52) U.S. Cl. 514/233.2; 544/344; 544/115; 514/250

(57) ABSTRACT

The present invention provides new compounds displaying high affinity for adenosine A₃ receptors. It also provides modulators of adenosine A₃ receptors. It further provides compounds for the treatment and/or prophylaxis of conditions and diseases where adenosine A₃ receptor plays a role. Pharmaceutical compositions for the treatment and/or prophylaxis of conditions and diseases where adenosine A₃ receptors play a role are also described.

The present invention provides new compounds displaying high affinity for adenosine A₃ receptors. It also provides modulators of adenosine A₃ receptors. It further provides compounds for the treatment and/or prophylaxis of conditions and diseases where adenosine A₃ receptor plays a role. Pharmaceutical compositions for the treatment and/or prophylaxis of conditions and diseases where adenosine A₃ receptors play a role are also described.

Adenosine is an ubiquitous modulator of numerous physiological activities, particularly within the cardiovascular and nervous systems. The effects of adenosine are mediated by specific cell surface receptor proteins. Adenosine modulates diverse physiological functions including induction of sedation, vasodilatation, suppression of cardiac rate and contractility, inhibition of platelet aggregability, stimulation of gluconeogenesis and inhibition of lipolysis. In addition to its effects on adenylate cyclase, adenosine has been shown to open potassium channels, reduce flux through calcium channels, and inhibit or stimulate phosphoinositide turnover through receptor-mediated mechanisms (Muller C. E. and Stein B., Current Pharmaceutical Design, 2:501, 1996, and Muller C. E., Exp. Opin. Ther. Patents, 7(5):419, 1997).

Adenosine receptors belong to the superfamily of purine receptors. Four major classes of adenosine receptors have been pharmacologically, structurally and functionally characterized (Fredholm et al., Pharm. Rev. (1994) 46:143-156) and classified A₁, A₂ₐ, A_{2B} and A₃ (referred to as A₁, A_{2A}, A_{2B} and A₃ receptors among the present application) A₁ receptors are coupled to the inhibition of adenylate cyclase through Gᵢ proteins and have also been shown to couple to other second messengers systems, including inhibition or stimulation of phosphoinositol turnover and activation of ion channels. A_{2A} and A_{2B} receptors are coupled to Gₛ proteins promoting the activation of adenylate cyclase, and leading to an increase in cellular CAMP levels. The A₁, A₂ₐ and A_{2B} subtypes were initially discovered by a study of agonist pharmacology but the A₃ subtype was recently discovered by molecular biology studies. In fact, the A₃ receptor sequence was first identified in a rat testes cDNA library, and this sequence was later cloned by homology to other G-protein coupled receptors (GPCRs) from a rat brain cDNA library.

In rat, the transcript of the A₃ receptor is found primarily in the central nervous system, testes, lung, kidneys, heart and inflammatory/immune cells. It seems to be a large interspecies difference in peripheral distribution of A₃ receptor among mammals: in the sheep, the transcript is especially found in the lung, spleen, pars tuberalis, pineal gland and inflammatory/immune cells, with lower levels in testis, kidneys and brain (Linden et al., Mol. Pharmacol. (1993), 44:524-532); the human transcript is widespread and the most abundant expression is detected in the lung and liver (Salvatore et al., Proc. Natl. Acad. Sci. USA (1993), 90:10365-10369). A₃ receptors have been shown to be involved in the pathophysiology of asthma and additional inflammatory conditions as chronic obstructive pulmonary disease (Meade C. J. et al., Life Science (2001), 69, 1225-1240; Polosa et al., Eur Respir. (2002), 20:488-496; Cronstein B. N. et al., Arthritis Rheum. (1995), 38, 1040-1045). A₃ receptors are also involved in the regulation of intraocular pressure (Yang et al., Current Eye Research 30 (2005), 747-754) and modulating them is a novel approach for treating glaucoma.

Further investigations have identified A₃ receptors in a number of human cancers, including pancreatic cancer, colon cancer, breast cancer, lung cancer and human malignant melanoma. Surprisingly, A₃ receptors are found at higher concentrations in the cancerous cells as compared to normal healthy tissue. In the patent U.S. Ser. No. 10/134,219 (Baraldi et al.) which is hereby incorporated by reference, the inventors demonstrated success in using A₃ receptor antagonists to induce apoptosis in human cancers. It shows that the use of A₃ antagonists allows targeting of the cancer cells for apoptosis, thereby reducing anticipated side effects in treatment of patients.

To date, IB-MECA (N′-(3-iodobenzyl)-adenosine-5′-N-methyluronamide) and CL-IB-MECA (2-chloro-N⁶ (3-iodobenzyl)-adenosine-5′-N-methyluronamide) (Jacobson et al., FEBS, 336(1), 57-60; Kim H. O. et al., Journal of Medicinal Chemistry, 1994, 37, 3614-3621) are the most potent and specific A₃ receptors agonists and have been widely used in a variety of studies. To show the interest of an A₃ receptor agonist an example is the prevention of ischemia. In fact, adenosine is released in large amounts during myocardial ischemia and can mediate potentially important protective functions in the cardiovascular system. Therefore, specific A₃ agonists can precondition the heart when given before the onset of ischemia and can cause a reduction of the consequences of the onset (Liang et al., patent U.S. Pat. No. 6,211,165). Agonists selective for the A₃ receptor are of interest as cerebroprotective (Jacobson et al., Trends Pharmacol. Sci. (1996), 17, 108-113), cardioprotective (Nakano et al., Pharmacol. Ther. (2000) 86, 263-275 and Dougherty et al., FASEB J. (1998) 12, 1785-1792) and anticancer agents (Fishman et al., Oncogene (2002), 21, 4060-4064).

Mast cell degranulation is a component of myocardial reperfusion injury, hypersensitivity reactions (asthma, allergic rhinitis, and urticaria), ischemic bowel disease, autoimmune inflammation, and atopic dermatitis. Selective A₃ receptor antagonists can be used to treat and/or prevent these diseases and generally the pathologic effects that result from mast cell degranulation. Specific A₃ receptor antagonists already identified (Jacobson et al., U.S. Pat. No. 6,376,521) are currently developed in several applications among which asthma, chronic obstructive pulmonary disease, glaucoma and other intraocular pressure troubles (Okamura et al., Bioorganic & Med Chem Letters, 14 (2004), 3775-3779). To date, A₃ receptor antagonists are sought to be useful as antiasthmatic, antidepressant, antiarrhythmic, renal protective, antiparkinson and cognitive enhancing drugs, but also as anti-inflammatory or possibly anti-ischemic agents in the brain.

Additional adenosine receptor modulators are needed as pharmacological tools and are of considerable interest as drugs for the treatment and/or prophylaxis of various diseases where A₃ receptors play a role. There remains a need for specific modulators of A₃ receptors. The present invention provides compounds with improved potency for A₃ receptor binding affinity and A₃ selectivity against other subtypes. The invention provides also methods of using these compounds to selectively modulate A₃ receptors in patients in need thereof, and pharmaceutical compositions comprising such compounds. These and other objects and advantages of the present invention, as well as additional inventive features, will be apparent from the description of the invention provided herein.

The present invention provides compounds of formula (I):

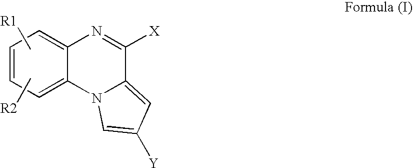

wherein:
R1 and R2 are independently from each other hydrogen, halogen, CN, CF₃, OCF₃, alkyl, COOH, O-(alkyl), S-(alkyl), N-(lower alkyl)(alkyl), heterocycloalkyl, aryl or heteroaryl, NH-(alkyl);
X represents R3 or COR3, O—R3 or S—R3, N—R3R4, NHCOR3, N(lower alkyl)COR3, NHSO₂R3, N(lower alkyl)SO₂R3 or NHCONHR3 with
   R3 and R4 are independently from the other hydrogen, alkyl, CF₃, aryl, heteroaryl, alkylaryl or alkylheteroaryl;
Y represents a (A)ₙ-B group wherein
   n represents either 0 or 1,
   A is a moiety selected in the group consisting of O, S, NH, N-(lower alkyl), N-aryl, N-heteroaryl,
   B is an aryl or a heteroaryl group;
      as well as pharmaceutically acceptable salt thereof, a prodrug of the compound or the salt, or a solvate or hydrate of the compound, the salt or the prodrug.

In a preferred embodiment R1 und R2 are independently from each other hydrogen, halogen, CN, CF₃, OCF₃, lower alkyl, COOH, O-(lower alkyl), S-(lower alkyl), N-(lower alkyl) (lower alkyl), heterocycloalkyl, aryl or heteroaryl.

In a further embodiment, the invention provides compounds of formula (II) as follows:

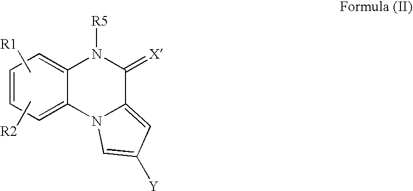

wherein:
R1, R2 and Y are as defined in formula (I);
X′ is selected from O or S
R5 is selected from alkyl, alkylaryl or alkylheteroaryl.

In a preferred embodiment R1 und R2 are independently from each other hydrogen, halogen, CN, CF₃, OCF₃, lower alkyl, COOH, O-(lower alkyl), S-(lower alkyl), N-(lower alkyl) (lower alkyl), heterocycloalkyl, aryl or heteroaryl.

As used herein, the term “alkyl” includes straight or branched chain saturated hydrocarbon residues with 1-8 carbon atoms, such as methyl, ethyl, n-propyl, isopropyl, n-butyl, i-butyl, s-butyl and t-butyl. These alkyls can be further substituted with groups such as COOH, heterocycloalkyl, CF₃, OH, O-(lower alkyl) or N-(lower alkyl)(lower alkyl).

As used herein, the term “lower alkyl” includes straight or branched chain saturated hydrocarbon residues with 1-4 carbon atoms, such as methyl, ethyl, n-propyl, isopropyl, n-butyl, i-butyl, s-butyl or t-butyl.

The term “aryl” refers to a 6-10 atoms aromatic hydrocarbon ring or fused aromatic hydrocarbon ring system containing at least one unsaturated aromatic ring. Examples of the term “aryl” are phenyl, naphthyl and 1,2,3,4-tetrahydronaphthyl. These aryls can be further substituted with groups such as halogen, CN, CF₃, OCF₃, lower alkyl, COOH, O-(lower alkyl), S-(lower alkyl), N-(lower alkyl)(lower alkyl) or heterocycloalkyl.

The term “heteroaryl” means a 5-10 atoms aromatic ring or fused aromatic rings containing one or more O, S, or N atoms. Examples of heteroaryls include pyridinyl, quinolinyl, isoquinolinyl, pyridazinyl, pyrimidinyl, pyrazinyl, furyl, benzofuryl, thienyl, benzothienyl, pyrrolyl, indolyl, pyrazolyl, indazolyl, oxazolyl, benzoxazolyl, thiazolyl, benzothiazolyl, imidazolyl, benzimidazolyl, and tetrazolyl. These heteroaryls can be further substituted with groups such as halogen, CN, CF₃, OCF₃, lower alkyl, COOH, O-(lower alkyl), S-(lower alkyl), N-(lower alkyl)(lower alkyl) or heterocycloalkyl.

The term “heterocycloalkyl” means a 4-7 atoms ring containing one or more O, S, or N atoms. Examples of heterocycloalkyls include azetidinyl, pyrrolidinyl, tetrahydrofuranyl, imidazolinyl, pyrrolidin-2-one, morpholinyl, thiomorpholinyl, piperidinyl, piperidin-2-one, piperazinyl, N-alkyl-piperazinyl.

The term “alkylaryl” means an alkyl-aryl-group or radical wherein alkyl and aryl have the meanings as defined above. Illustrative examples of an alkylaryl group or radical include benzyl, 2-phenylethyl, 3-phenylpropyl, 4-phenylbutyl, 3-methyl-3-phenylpropyl, 1-naphthylmethyl, 1-naphthylethyl.

The term “alkylheteroaryl” means an alkyl-heteroaryl-group or radical wherein alkyl and heteroaryl have the meanings as defined above.

The term “halogen” refers to bromine, chlorine, fluorine, or iodine.

Pharmaceutically acceptable salts of compounds of formula (I) include salts with inorganic or organic acids, e.g. hydrochloric, hydrobromic, nitric, carbonic, formic, monohydrogencarbonic, phosphoric, monohydrogenphosphoric, dihydrogenphosphoric, perchloric, sulfuric, monohydrogensulfuric, hydroiodic, phosphorous, acetic, lactic, propionic, butyric, isobutyric, palmoic, maleic, glutamic, hydroxymaleic, malonic, benzoic, succinic, glycolic, suberic, fumaric, mandelic, phthalic, salicylic, benzenesulfonic, p-tolylsulfonic, citric, tartaric, methanesulfonic and hydroxynaphthoic acids.

Pharmaceutically acceptable salts of compounds of formula (I) can also include salts with inorganic bases, e.g. alkali metal bases, especially sodium or potassium bases or alkaline-earth metal bases, especially calcium or magnesium bases, or with pharmaceutically acceptable organic bases.

The invention is based, at least in part, on the discovery that the compound described above can be used to prevent and/or treat various medical disorders and/or conditions where A₃ receptors play a role. These disorders and/or conditions are associated with, for example, asthma, hypersensitivity, rhinitis, hay fever, serum sickness, allergic vasculitis, atopic dermantitis, dermantitis, psorasis, eczema, idiopathic pulmonary fibrosis, eosinophillic chlorecystitis, chronic airway inflammation, chronic obstructive pulmonary disease, hypereosinophilic syndromes, eosinophilic gastroenteritis, edema, urticaria, eosinophilic myocardial disease, episodic angioedema with eosinophilia, inflammatory bowel disease, ulcerative colitis, allergic granulomatosis, carcinomatosis, eosinophilic granuloma, familial histiocytosis, hypertension, mast cell degranulation, tumor, cardiac hypoxia, cerebral ischemia, diuresis, renal failure, neurological disorder, mental disorder, cognitive disorder, myocardial ischemia, bronchoconstriction, arthritis, autoimmune disease, Crohn's disease, Grave's disease, diabetes, multiple sclerosis, anaemia, psoriasis, fertility disorders, lupus erthyematosus, reperfusion injury, brain arteriole diameter, the release of allergic mediators, scleroderma, stroke, global ischemia, central nervous system disorder, cardiovascular disorder, renal disorder, inflammatory disorder, gastrointestinal disorder, eye disorder, allergic disorder, respiratory disorder, or immunological disorder.

Among preferred compounds of formula (I), preferred embodiments include those wherein R1, R2 and X are as defined in formula (I) and Y represents an aryl group.

Further preferred embodiments include compounds of formula (I) wherein R1 is hydrogen, R2 and X is as defined in formula (I) and Y represents an aryl group.

Other preferred compounds of formula (I) are those wherein R1 and R2 are hydrogen, X is as defined in formula (I) and Y represents an aryl group.

Additional preferred compounds of formula (I) are those wherein R1 and R2 are hydrogen, X represents R3, O—R3 or S—R3, N—R3R4, with:
R3 and R4 are independently from the other hydrogen, alkyl, CF₃, aryl, heteroaryl, alkylaryl or alkylheteroaryl;
   and Y represents an aryl group.

The compounds of general formula (I) and their pharmaceutically acceptable salts can be manufactured according to methods described in the following schemes:

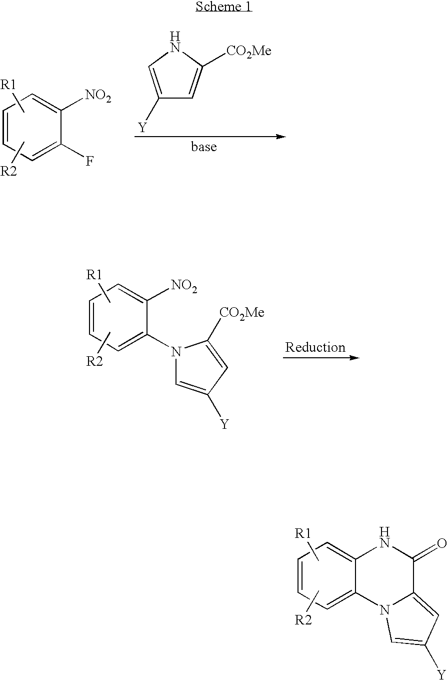

Compounds of general formula (I) were synthesized according to scheme 1.

In a first step, a pyrrole moiety was introduced on 2-fluoro-3-nitrobenzyl derivatives by nucleophilic substitution. Then, a one-pot reduction-cyclization step afforded the tricyclic compounds.

When Y is an aromatic substituent, it is introduced via a cross-coupling reaction with the corresponding aryl boronic acid:
On the pyrrole ring prior to the nucleophilic substitution step.
On the bicyclic structure before the reduction step.
On the tricyclic structure after the reduction step.

Substituents R1 and R2 are introduced—or modified—on the benzyl moiety by state of the art procedures either before or after the nucleophilic substitution step.

Further diversity on the tricyclic structures is introduced according to scheme 2:

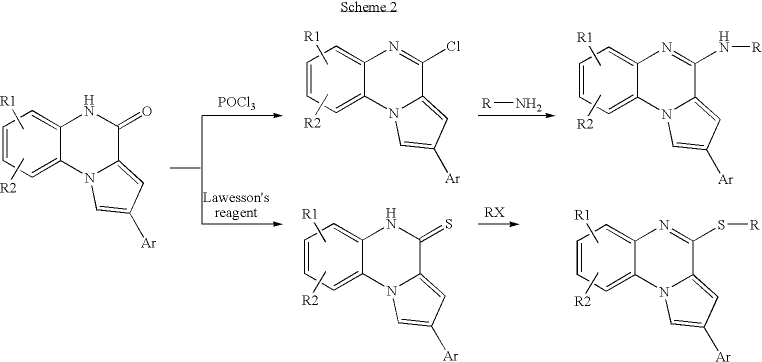

The present invention pertains to methods for modulating A₃ receptor functioning by the administration of a therapeutically effective amount of a compound of general formula (I) to a patient in need thereof, such that modulation of the adenosine receptor's activity occurs.

The modulation of the adenosine receptor's activity can be assessed in binding assays which are well known to one skilled in the art. The following methods are widely used: for A₁ receptor the procedure described by Townsend-Nicholson and Schofield (J. Biol. Chem. (1994), 269:2373-2376), for A_{2A} receptor the procedure described by Luthin et al. (Mol. Pharmacol. (1995), 47:307-313), for A_{2B} receptor the procedure described by Stehle et al. (Mol. Endocrinol. (1992), 6:384-393), and for A₃ receptor the procedure described by Salvatore et al. (Proc. Natl. Acad. Sci. (1993), 90:10365-10369).

In a preferred embodiment, the compound of formula (I) is an A₃ receptor antagonist.

In a further embodiment, the invention provides methods of selectively blocking A₃ receptor in a mammal by administration of a therapeutically effective amount of a compound of formula (I) to a patient in need thereof, such that blockage of the adenosine receptor's activity occurs.

Preferred compounds of formula (I) according to the invention are:
2-Phenyl-5H-pyrrolo[1,2-a]quinoxalin-4-one.

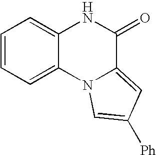

2-(4-Methoxy-phenyl)-5H-pyrrolo[1,2-a]quinoxalin-4-one.

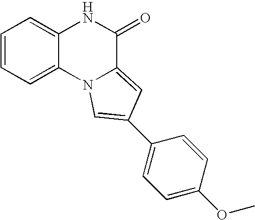

8-Methyl-2-phenyl-5H-pyrrolo[1,2-a]quinoxalin-4-one.

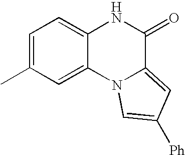

8-Chloro-2-phenyl-5H-pyrrolo[1,2-a]quinoxalin-4-one.

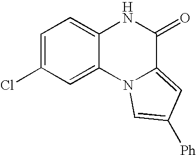

8-(4-Methyl-piperazin-1-yl)-2-phenyl-5H-pyrrolo[1,2-a]quinoxalin-4-one.

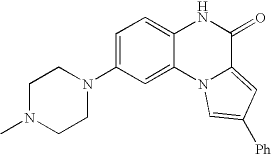

8-(3-Morpholin-4-yl-propylamino)-2-phenyl-5H-pyrrolo[1,2-a]quinoxalin-4-one.

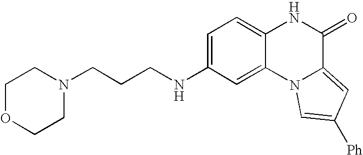

7-Morpholin-4-yl-2-phenyl-5H-pyrrolo[1,2-a]quinoxalin-4-one.

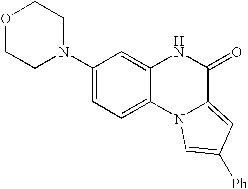

8-(2-Morpholin-4-yl-ethoxy)-2-phenyl-5H-pyrrolo[1,2-a]quinoxalin-4-one.

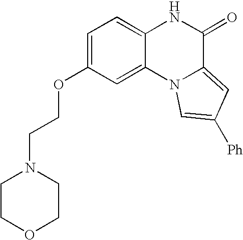

The invention provides also the use of a compound of general formula (I) in the manufacture of a drug for use in the treatment and/or prophylaxis of conditions and/or disorders where A₃ receptors play a role.

The present invention further provides a pharmaceutical composition comprising a pharmaceutically effective amount of a compound of the formula (I), or pharmaceutically acceptable salts thereof, in combination with a pharmaceutically acceptable carrier, diluent or excipient.

According to the invention, the terms “treatment and/or prophylaxis” refer to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder, improvement of the patient's quality of life.

In another embodiment, the invention provides a pharmaceutical composition for the treatment and/or prophylaxis of a disorders and/or or condition where A₃ receptors play a role, associated with, for example, asthma, hypersensitivity, rhinitis, hay fever, serum sickness, allergic vasculitis, atopic dermantitis, dermantitis, psorasis, eczema, idiopathic pulmonary fibrosis, eosinophillic chlorecystitis, chronic airway inflammation, chronic obstructive pulmonary disease, hypereosinophilic syndromes, eosinophilic gastroenteritis, edema, urticaria, eosinophilic myocardial disease, episodic angioedema with eosinophilia, inflammatory bowel disease, ulcerative colitis, allergic granulomatosis, carcinomatosis, eosinophilic granuloma, familial histiocytosis, hypertension, mast cell degranulation, tumor, cardiac hypoxia, cerebral ischemia, diuresis, renal failure, neurological disorder, mental disorder, cognitive disorder, myocardial ischemia, bronchoconstriction, arthritis, autoimmune disease, Crohn's disease, Grave's disease, diabetes, multiple sclerosis, anaemia, psoriasis, fertility disorders, lupus erthyematosus, reperfusion injury, brain arteriole diameter, the release of allergic mediators, scleroderma, stroke, global ischemia, central nervous system disorder, cardiovascular disorder, renal disorder, inflammatory disorder, gastrointestinal disorder, eye disorder, allergic disorder, respiratory disorder, or immunological disorder.

The compounds of the invention may be administered in a form suitable for oral use, for example a tablet, capsule, aqueous or oily solution, suspension or emulsion; for topical use including transmucosal and transdermal use, for example a cream, ointment, gel, aqueous solution or suspension, salve, patch or plaster; for nasal use for example a snuff, nasal spray or nasal drops; for vaginal or rectal use, for example a suppository; for administration by inhalation, for example a finely divided powder or a liquid aerosol; for sub-lingual or buccal use, for example a tablet or a capsule; or for parenteral use (including intravenous, subcutaneous, intramuscular, intravascular or infusion), for example a sterile aqueous solution or suspension, or depot injection formulation. In general, the above compositions may be prepared in a conventional manner using well known excipients, using standard techniques, including controlled release technologies, such as gelatin, lipid, gel depot, liposome and/or microcapsule based systems well known to those skilled in the art of pharmacy.

For an oral administration, the compounds of the invention will generally be provided in the form of tablets or capsule or as an aqueous solution or suspension.

Tablets for oral use may include the active ingredient mixed with pharmaceutically acceptable excipients such as inert diluents (such as sodium and calcium carbonate, sodium and calcium phosphate, or lactose), disintegrating agents (such as corn starch or alginic acid), binding agents (starch or gelatin), lubricating agents (magnesium stearate, stearic acid or talc), sweetening agents, flavoring agents, coloring agents and preservatives. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredient is mixed with water or oil such as peanut oil, liquid paraffin or olive oil.

For intramuscular, intraperitoneal, subcutaneous and intravenous use, the compounds of the invention will generally be provided in sterile aqueous solutions (such as Ringer's solution or isotonic sodium chloride) or suspensions (such as cellulose derivatives, sodium alginate, polyvinylpyrrolidone and gum tragacanth with a wetting agent such as lecithin, and a preservative such as ethyl and n-propyl p-hydroxybenzoate), buffered to an appropriate pH and isotonicity.

Transdermal formulations include membrane permeation systems, multi-laminate adhesive dispersion systems and matrix dispersion systems. Transdermal delivery also includes the use of electrically aided transport and skin penetration enhancers.

The preferred route of administration is the intravenous infusion, preferably over a period of up to seven days, or as an oral formulation, or as an intramuscular injection via a styrette or as a subcutaneous injection.

It will be appreciated that the dosage levels used may vary over quite a wide range depending upon the compound used, the severity of the condition exhibited by the patient and the patient's body weight. However, without commitment to a rigid definition of dosages, it may be stated that a daily dosage of the active ingredient is 0.01 to about 100 mg/kg body weight of the patient being treated per day. Preferred dosages range from about 0.1 to about 10 mg/kg body weight/day.

Other characteristics and advantages of the invention are given in the following examples in which reference is made to:

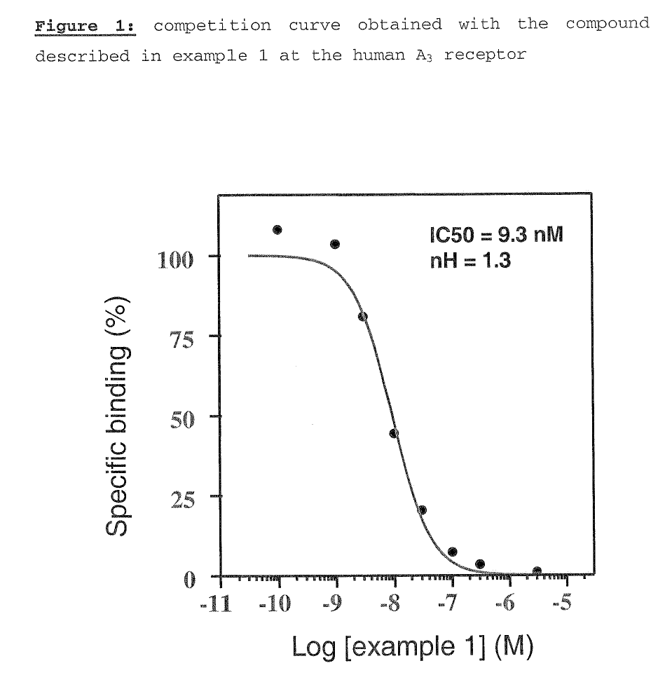

FIG. 1 which represents the competition curve obtained with the compound described in example 1 at the human A₃ receptor;

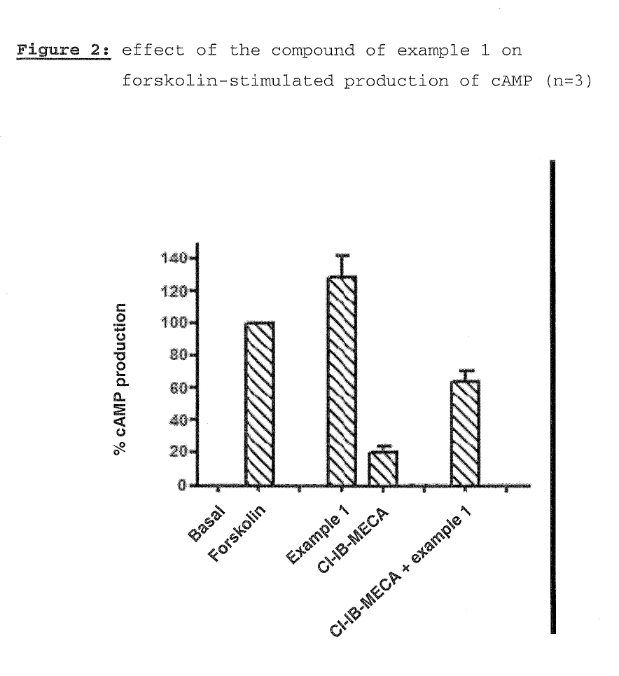

FIG. 2 which represents the effect of compound of the example 1 on forskolin-stimulated production of cAMP

It will be appreciated that the invention is described by way of example only and modification of detail may be made without departing from the scope of the invention.

### EXAMPLES

### Preparation A: 3-Bromo-1-(2-nitro-phenyl)-1H-pyrrole-2-carboxylic acid methyl ester

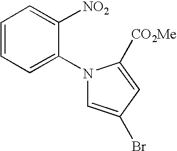

To a solution of methyl-4-bromo-pyrrole-2-carboxylate (5.0 g, 1.0 equiv) in DMF (45 mL) was added cesium carbonate (9.0 g, 1.2 equiv). The mixture was stirred at R.T. for 10 min, then 1-fluoro-2-nitrobenzene (3.9 mL, 1.5 equiv) was added. The reaction mixture was heated through microwave irradiation 5 min at 130° C. The reaction mixture was then diluted in AcOEt, washed with HCl 1M, water, brine and dried over MgSO₄. The organic layer was concentrated to give a dark brown oil. After triturating in Et₂O a solid was obtained and was filtered off, washed with Et₂O and dried to give the desired product as a yellow solid in 88% yield.

¹H-NMR (400 MHz, D6-DMSO): 3.59 (s, 3H, CH₃—O); 7.12 (d, J 1.9 Hz, 1H, Ar); 7.57 (d, J 1.9 Hz, 1H, Ar); 7.65 (dd, J 1.8 Hz, J 7.8 Hz, 1H, Ar); 7.76 (td, J 1.4 Hz, J 7.8 Hz, 1H, Ar); 7.87 (td, J 1.5 Hz, J 7.7 Hz, 1H, Ar); 8.21 (dd, J 1.5 Hz, J 8.1 Hz, 1H, Ar).

M/Z (M+H)⁺=327.

### Preparation B: 2-Bromo-5H-pyrrolo[1,2-a]quinoxalin-4-one

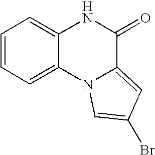

A suspension of compound from preparation A (6.4 g, 1 equiv) and iron powder (4.5 g, 4.0 equiv) in acetic acid (25 mL) was heated through microwave irradiation 8 min at 150° C. A dark solid was obtained and was suspended in HCl 1M, filtered off, washed with HCl 1M, AcOEt, Et₂O and dried to give a fluffy white solid in 90% yield.

¹H-NMR (400 MHz, D6-DMSO): 7.09 (d, J 1.5 Hz, 1H, Ar); 7.22 (t, J 7.0 Hz, 1H, Ar); 7.30 (m, 2H, Ar); 8.05 (d, J 8.0 Hz, 1H, Ar); 8.43 (d, J 1.6 Hz, 1H, Ar); 11.45 (bs, 1H, NH).

M/Z (M+H)⁺=265.

### Preparation C: 1-(2-Nitro-phenyl)-4-phenyl-1H-pyrrole-2-carboxylic acid methyl ester

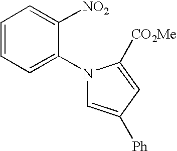

A mixture of compound from preparation A (2.50 g, 1 equiv), phenylboronic acid (1.87 g, 2 equiv), sodium carbonate (2.45 g, 3 equiv) and Pd(PPh₃)₄ (50 mg, 10%) in methanol/water (4:1) (25 mL) was heated through microwave irradiation 30 min at 150° C. (Pₘₐₓ 70 W). The reaction mixture was then diluted with AcOEt and washed with HCl 1M, water, brine and dried over MgSO₄. The organic layer was concentrated to give a brown oil. Purification by flash chromatography (5% to 15% AcOEt in cyclohexane) afforded a yellow solid that was triturated with Et₂O and dried to give the desired product in 22% yield.

¹H-NMR (400 MHz, D6-DMSO): 2.97 (s, 3H, CH₃—O); 7.23 (t, J 7.4 Hz, 1H, Ar); 7.37 (t, J 7.6 Hz, 2H, Ar); 7.50 (d, J 2.0 Hz, 1H, Ar); 7.70 (d, J 7.9 Hz, 3H, Ar); 7.77 (td, J 1.4 Hz, J 7.7 Hz, 1H, Ar); 7.88 (m, 2H, Ar); 8.21 (dd, J 1.4 Hz, J 8.1 Hz, 1H, Ar).

M/Z (M+H)⁺=323.

### Preparation D: 3-(4-Methoxy-phenyl)-1-(2-nitro-phenyl)-1H-pyrrole-2-carboxylic acid methyl ester

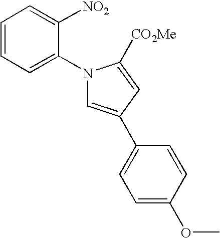

Compound obtained following the procedure described in preparation C from compound from preparation A and 4-methoxyphenylboronic acid in 54% yield as a yellow solid.

¹H-NMR (400 MHz, D6-DMSO): 3.61 (s, 3H, COOCH₃); 3.77 (s, 3H, CH₃O); 6.94 (dt, J 2.2 Hz, J 8.9 Hz, 2H, Ar); 7.43 (d, J 2.0 Hz, 1H, Ar); 7.62 (dt, J 2.2 Hz, J 8.9 Hz, 2H, Ar); 7.68 (dd, J 1.3 Hz, J 7.8 Hz, 1H, Ar); 7.73-7.78 (m, 2H, Ar); 7.88 (td, J 1.4 Hz, J 7.7 Hz, 1H, Ar); 8.20 (dd, J 1.4 Hz, J 8.1 Hz, 1H, Ar). M/Z (M+H)⁺=353.

### Preparation E: 4-Chloro-2-phenyl-pyrrolo[1,2-a]quinoxaline

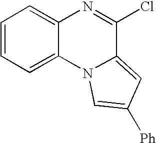

A suspension of compound from example 1 (500 mg, 1 equiv) in phosphorus oxychloride (10 mL) was heated through microwave irradiation 10 min at 120° C. A yellow solid appeared and was filtered off, washed with CH₂Cl₂ and dried. The solid was then dissolved in DMF and re-precipitated by addition of water. The resulting white solid was filtered off, washed with Et₂O and dried to give the desired product in 36% yield.

¹H-NMR (400 MHz, D6-DMSO): 7.33 (t, J 7.2 Hz, 1H, Ar); 7.51 (m, 4H, Ar); 7.70 (t, J 7.8 Hz, 1H, Ar); 7.86 (dd, J 1.2 Hz, J 8.0 Hz, 1H, Ar); 7.92 (dd, J 1.2 Hz, J 8.2 Hz, 2H, Ar); 8.38 (dd, J 1.2 Hz, J 8.2 Hz, 1H, Ar); 9.16 (d, J 1.5 Hz, 1H, Ar).

M/Z (M+H)⁺=279.

### Preparation F: 4-Bromo-1-(5-fluoro-2-nitro-phenyl)-1H-pyrrole-2-carboxylic acid methyl l ester

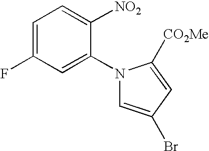

A suspension of 2,4-difluoronitrobenzene (448 μL, 1 equiv), methyl-4-bromopyrrole-2-carboxylate (1.0 g, 1.2 equiv) and cesium carbonate (1.2 g, 1 equiv) in DMF (10 mL) was heated through microwave irradiation 10 min at 100° C. The reaction mixture was then diluted in AcOEt, washed with HCl 1M, water, brine and dried over MgSO₄. The organic layer was concentrated to give a yellow oil. Purification by flash chromatography (5% to 20% AcOEt in cyclohexane) afforded a yellow solid in 30% yield.

¹H-MMR (400 MHz, D6-DMSO): 3.62 (s, 3H, COOCH₃); 7.13 (d, J 1.9 Hz, 1H, Ar); 7.60 (d, J 1.9 Hz, 1H, Ar); 7.66 (m, 1H, Ar); 7.77 (dd, J 2.7 Hz, J 12.8 Hz, 1H, Ar); 8.33 (dd, J 5.4 Hz, J 9.2 Hz, 1H, Ar).

M/Z (M+H)⁺=345.

### Preparation G: 4-Bromo-1-(5-dimethylamino-2-nitro-phenyl)-1H-pyrrole-2-carboxylic acid methyl ester

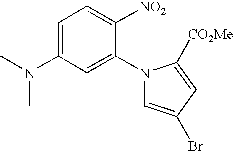

A mixture of compound from preparation F, diisopropylethylamine (50 μL, 1 equiv) and dimethylamine 2M solution in THF (1.5 mL) was heated through microwave irradiation 5 min at 100° C. The reaction mixture was then diluted in AcOEt, washed with HCl 1M, water, brine and dried over MgSO₄. The organic layer was concentrated to give a light orange solid in a quantitative yield.

¹H-NMR (400 MHz, D6-DMSO): 3.10 (s, 6H, N(CH₃)₂); 3.60 (s, 3H, COOCH₃); 6.67 (d, J 2.8 Hz, 1H, Ar); 6.84 (dd, J 2.8 Hz, J 9.5 Hz, 1H, Ar); 7.05 (d, J 1.9 Hz, 1H, Ar); 7.44 (d, J 1.9 Hz, 1H, Ar); 8.12 (d, J 9.5 Hz, 1H, Ar).

M/Z (M+H)⁺=368.

### Preparation H: 2-Bromo-8-dimethylamino-5H-pyrrolo[1,2-a]quinoxalin-4-one

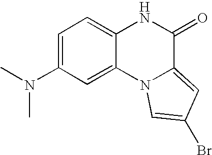

Compound obtained from compound from preparation G following the procedure described in preparation B in 80% yield as white needles.

¹H-NMR (400 MHz, D6-DMSO): 3.00 (s, 6H, N(CH₃)₂); 6.93 (b, 1H, Ar); 7.05 (d, J 1.7 Hz, 1H, Ar); 7.20 (d, J 8.9 Hz, 1H, Ar); 7.46 (b, 1H, Ar); 8.50 (d, J 1.8 Hz, 1H, Ar); 11.26 (bs, 1H, NH).

M/Z (M+H)⁺=306.

### Preparation I: 4-Bromo-1-(5-morpholin-4-yl-2-nitro-phenyl)-1H-pyrrole-2-carboxylic acid methyl ester

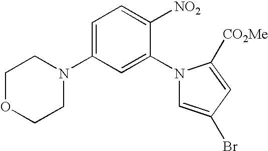

A mixture of compound from preparation F, morpholine (61 μL, 1.2 equiv) and diisopropylethylamine (122 μL, 1 equiv) in DMF (1 mL) was heated through microwave irradiation 5 min at 100° C. The reaction mixture was then diluted in AcOEt, washed with HCl 1M, water, brine and dried over MgSO₄. The organic layer was concentrated to give a yellow solid. Purification by flash chromatography (10% to 50% AcOEt in cyclohexane) afforded the desired product as a yellow solid in 80% yield.

¹H-NMR (400 MHz, D6-DMSO): 3.45 (m, 4H, N(CH₂)₂); 3.60 (s, 3H, COOCH₃); 3.71 (m, 4H, O(CH₂)₂); 6.99 (d, J 2.8 Hz, 1H, Ar); 7.07 (dd, J 2.8 Hz, J 9.5 Hz, 1H, Ar); 7.11 (d, J 2.8 Hz, 1H, Ar); 7.45 (d, J 2.0 Hz, 1H, Ar); 8.13 (d, J 9.5 Hz, 1H, Ar).

M/Z (M+H)⁺=410.

### Preparation J: 1-(5-Morpholin-4-yl-2-nitro-phenyl)-4-phenyl-1H-pyrrole-2-carboxylic acid methyl ester

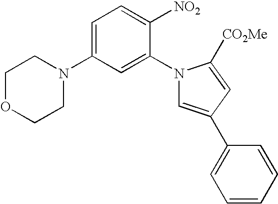

Compound obtained from compound from preparation I following procedure described in preparation C as a yellow solid in 60% yield.

¹H-NMR (400 MHz, D6-DMSO): 3.45 (m, 4H, N(CH₂)₂); 3.62 (s, 3H, COOCH₃); 3.72 (m, 4H, O(CH₂)₂); 7.02 (d, J 2.7 Hz, 1H, Ar); 7.11 (dd, J 2.8 Hz, J 9.5 Hz, 1H, Ar); 7.21 (m, 1H, Ar); 7.36 (m, 2H, Ar); 7.45 (d, J 2.0 Hz, 1H, Ar); 7.68 (m, 2H, Ar); 7.75 (d, J 2.0 Hz, 1H, Ar); 8.14 (d, J 9.5 Hz, 1H, Ar).

M/Z (M+H)⁺=408.

### Preparation K: 4-(4-tert-Butoxycarbonylamino-phenyl)-1-(2-nitro-phenyl)-1H-pyrrole-2-carboxylic acid methyl ester

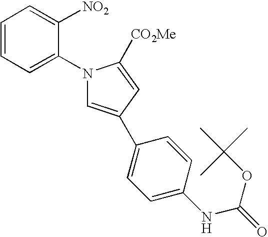

Compound obtained following the procedure described in preparation C from compound from preparation A and tert-butyl-N-[4-(4,4,5,5-tetramethyl-1,3,2-dioxaborolan-2-yl)phenyl]carbamatemethoxyphenylboronic acid in 44% yield as a yellow solid.

¹H-NMR (400 MHz, D6-DMSO): 1.49 (s, 9H, OOC(CH₃)₃); 3.61 (s, 3H, COOCH₃); 7.45 (m, 3H, Ar); 7.58 (m, 2H Ar); 7.68 (dd, J 1.4 Hz, J 7.8 Hz, 1H, Ar); 7.75 (m, 2H, Ar); 7.88 (td, J 1.5 Hz, J 7.7 Hz, 1H, Ar); 8.20 (dd, J 1.4 Hz, J 8.1 Hz, 1H, Ar); 9.37 (bs, 1H, NH).

M/Z (M+H—C₄H₈)⁺=382.

### Preparation L: 4-(4-Dimethylamino-phenyl)-1-(2-nitro-phenyl)-1H-pyrrole-2-carboxylic acid methyl ester

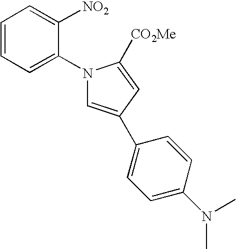

A mixture of compound from preparation K (150 mg, 1 equiv), formaldehyde 40% in aqueous solution (350 μL), sodium triacetoxyborohydride (210 mg, 3.2 equiv) and acetic acid (30 μL) in acetonitrile (1.5 mL) was stirred at R.T. After 2H, NaBH(OAc)₃ (105 mg) and formaldehyde (175 μL) were added and the reaction mixture was stirred for a further 2H. The reaction mixture was then diluted in AcOEt, washed with a saturated aqueous solution of NaHCO₃, brine, and dried over MgSO₄. The organic layer was concentrated to give an orange solid in a quantitative yield.

¹H-NMR (400 MHz, D6-DMSO): 2.90 (s, 6H, N(CH₃)₂); 3.30 (s, 3H, COOCH₃); 6.73 (m, 2H, Ar); 7.36 (d, J 2.0 Hz, 1H Ar); 7.50 (m, 2H, Ar); 7.66 (m, 2H, Ar); 7.74 (m, 1H, Ar); 7.87 (td, J 1.5 Hz, J 7.7 Hz, 1H, Ar); 8.19 (d, J 1.4 Hz, 1H, Ar).

M/Z (M+H)⁺=366.

The following examples of compounds of formula (I) were obtained using above described routes of synthesis.

### Preparation M: 4-Bromo-pyrrole-1,2-dicarboxylic acid 1-tert-butyl ester 2-methyl ester

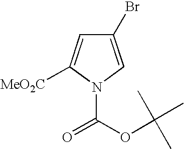

To a solution of 4-bromo-1H-pyrrole-2-carboxylic acid methyl ester (9.68 g, 1.0 equiv) in acetonitrile (100 mL), di-tert-butyl-dicarbonate (13.4 g, 1.3 equiv) and 4-dimethylaminopyridine (578 mg, 0.1 equiv) were added and the resulting mixture was stirred at R.T. for 2 Hrs. The reaction mixture was diluted with AcOEt (300 mL) and was washed with a 1M aqueous solution of KHSO₄ (2*150 mL) water (150 mL), a saturated aqueous solution of NaHCO₃ (150 mL), and brine (150 mL). The organic layer was dried over MgSO₄ and was concentrated under reduced pressure to give the product as a yellow oil in a quantitative yield.

¹H-NMR (400 MHz, D6-DMSO): 1.52 (s, 9H, tBu); 3.78 (s, 3H, OCH₃); 6.96 (d, J 1.7 Hz, 1H, Ar); 7.65 (d, J 1.7 Hz, 1H, Ar).

M/Z (M+H)⁺=218.

### Preparation N: 4-Phenyl-1H-pyrrole-2-carboxylic acid methyl ester

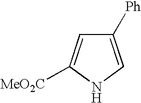

To a solution of 4-bromo-pyrrole-1,2-dicarboxylic acid 1-tert-butyl ester 2-methyl ester (19.4 g, 1.0 equiv) in methanol (500 mL), phenylboronic acid (13.6 g, 2.0 equiv), sodium methoxide (6.0 g, 2.0 equiv) and Pd(PPh₃)₄ (2.0 g, 3%) were added. The resulting mixture was heated at 75° C. under nitrogen stream for 4 days. The reaction mixture was concentrated to half its initial volume, and was then diluted with AcOEt (500 mL) and water (500 mL). The catalyst was filtered off on celite. The filtrate layers were separated and the organic layer was washed with water (500 mL) and brine (300 mL). The organic layer was dried over MgSO₄ and preabsorbed on silica. Purification by flash chromatography (AcOEt 5-30% in cyclohexane) afforded the product as a white solid in 80% yield.

¹H-NMR (400 MHz, D6-DMSO): 3.79 (s, 3H, OCH₃); 7.17 (m, 2H, Ar); 7.33 (m, 2H, Ar); 7.52 (m, 1H, Ar); 7.62 (m, 2H, Ar); 12.09 (s, 1H, NH).

M/Z (M+H)⁺=202.

### Preparation O: 1-(5-Methyl-2-nitro-phenyl)-4-phenyl-1H-pyrrole-2-carboxylic acid methyl ester

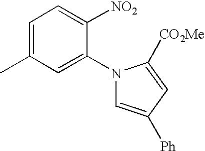

A mixture of 2-fluoro-4-methyl-1-nitro-benzene (288 mg, 1.5 equiv), 4-phenyl-1H-pyrrole-2-carboxylic acid methyl ester (250 mg, 1.0 equiv) and cesium carbonate (488 mg, 1.5 equiv) in dimethylformamide (5 mL) was heated through microwave irradiation for 30 min at 150° C. The reaction mixture was diluted with AcOEt (20 mL), washed with water (40+2*20 mL) and brine (20 mL). A solid precipitated from the organic layer, it was filtrated and dried under vacuum to give the product in 78% yield.

M/Z (M+H)⁺=337.

### Preparation P: 1-(5-Chloro-2-nitro-phenyl)-4-phenyl-1H-pyrrole-2-carboxylic acid methyl ester

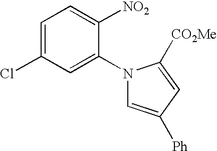

A mixture of 4-chloro-2-fluoronitrobenzene (228 mg, 1.05 equiv), 4-phenyl-1H-pyrrole-2-carboxylic acid methyl ester (250 mg, 1.0 equiv) and cesium carbonate (425 mg, 1.05 equiv) in dimethylformamide (9 mL) was heated through microwave irradiation for 5 min at 130° C. The reaction mixture was diluted with AcOEt (20 mL), washed with water (3*20 mL) and brine (20 mL). The organic layer was dried over MgSO₄ and concentrated under reduced pressure. The residue was triturated in Et₂O, filtered off, washed with Et₂O (5 mL) and dried under vacuum to give the product as a yellow solid in 75% yield.

¹H-NMR (400 MHz, D6-DMSO): 3.64 (s, 3H, OCH₃); 7.24 (t, J 7.3, 1H, Ar); 7.38 (m, 2H, Ar); 7.52 (d, J 2.0 Hz, 1H, Ar); 7.70 (dd, J 1.2 Hz, J 8.4 Hz, 2H, Ar); 7.87 (dd, J 2.4 Hz, J 8.6 Hz, 1H, Ar); 7.90 (d, J 2.4 Hz, 1H, Ar) 7.96 (d, J 2.4 Hz, 1H, Ar); 8.25 (d, J 8.6 Hz, 1H, Ar).

M/Z (M+H)⁺=357.

### Preparation Q: 1-[5-(4-Methyl-piperazin-1-yl)-2-nitro-phenyl]-4-phenyl-1H-pyrrole-2-carboxylic acid methyl ester

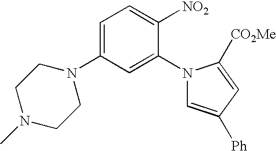

A mixture of 1-(5-chloro-2-nitro-phenyl)-4-phenyl-1H-pyrrole-2-carboxylic acid methyl ester (225 mg, 1.0 equiv), N-methylpiperazine (210 μL, 3.0 equiv) and DIPEA (110 μL, 1.0 equiv) in dimethylsulfoxide (5 mL) was heated at 110° C. for 16 Hrs. The reaction mixture was cooled to R.T and was diluted with AcOEt (15 mL), washed with water (3*10 mL) and brine (10 mL). The organic layer was dried over MgSO₄ and concentrated under reduced pressure. The residue was triturated in Et₂O, filtered off, washed with MeOH (5 mL) and dried under vacuum to give the product as a yellow solid in 73% yield.

¹H-NMR (400 MHz, D6-DMSO): 2.22 (s, 3H, NCH₃); 2.42 (m, 4H, 2 NCH₂); 3.48 (m, 4H, 2 NCH₂); 3.62 (s, 3H, OCH₃); 7.00 (d, J 2.7 Hz, 1H, Ar); 7.10 (dd, J 2.7 Hz, J 9.5 Hz, 1H, Ar); 7.21 (t, J 7.4 Hz, 1H, Ar); 7.36 (t, J 7.7 Hz, 2H, Ar); 7.45 (d, J 1.7 Hz, 1H, Ar); 7.69 (d, J 7.7 Hz, 2H, Ar); 7.76 (d, J 1.4 Hz, 1H, Ar); 8.12 (d, J 9.1 Hz, 1H, Ar).

M/Z (M+H)⁺=421.

### Preparation R: 1-[5-(3-Morpholin-4-yl-propylamino)-2-nitro-phenyl]-4-phenyl-1H-pyrrole-2-carboxylic acid methyl ester

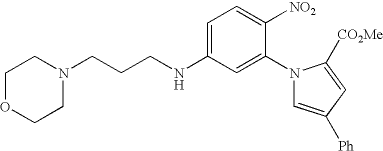

A mixture of 1-(5-chloro-2-nitro-phenyl)-4-phenyl-1H-pyrrole-2-carboxylic acid methyl ester (150 mg, 1.0 equiv), 3-morpholin-4-yl-propylamine (185 μL, 3.0 equiv) and DIPEA (75 μL, 1.0 equiv) in dimethylsulfoxide (4 mL) was heated at 110° C. for 6 Hrs. The reaction mixture was cooled to R.T. and was diluted with AcOEt (10 mL), washed with water (3*10 mL) and brine (10 mL). The organic layer was dried over MgSO₄ and concentrated under reduced pressure. Purification by flash chromatography (1% MeOH in CH₂Cl₂) gave the product as a green oil in 60% yield.

¹H-NMR (400 MHz, D6-DMSO): 2.33 (m, 6H, 3 CH₂); 3.17 (m, 2H, NCH₂); 3.23 (m, 2H, NCH₂); 3.52 (m, 4H, 2 OCH₂); 3.63 (s, 3H, OCH₃); 6.58 (d, J 2.7 Hz, 1H, Ar); 6.74 (dd, J 2.7 Hz, J 9.3 Hz, 1H, Ar); 7.21 (m, 1H, Ar); 7.36 (m, 2H, Ar); 7.43 (m, 2H, Ar+NH); 7.68 (dd, J 1.3 Hz, J 8.5 Hz, 2H, Ar); 7.74 (d, J 2.2 Hz, 1H, Ar); 8.08 (d, J 9.3 Hz, 1H, Ar).

M/Z (M+H)⁺=465.

### Preparation S: (3-Fluoro-4-nitro-phenyl)-acetic acid methyl ester

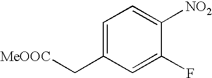

A solution of (3-fluoro-phenyl)-acetic acid methyl ester (26.0 g, 1.0 equiv) in H₂SO₄ (96%, 41 mL, 5.0 equiv) under nitrogen stream was cooled at 0° C., then fuming HNO₃ (6.6 mL, 0.9 equiv) was added slowly. The mixture was stirred at 0° C. for 1.5 Hrs, then was poured onto a water and ice mixture (250 mL) and vigorously stirred for 15 min. It was extracted with AcOEt (2*100 mL), the organic layer was washed with water (100 mL), brine (100 mL), dried over MgSO₄ and concentrated under reduced pressure. Purification by flash chromatography (15-20% AcOEt in cyclohexane) gave the desired isomer as a yellow oil in 11% yield.

¹H-NMR (400 MHz, D6-DMSO): 3.64 (s, 3H, OCH₃); 3.90 (s, 2H, CH₂); 7.37 (d, J 8.6 Hz, 1H, Ar); 7.54 (dd, J 1.7 Hz, J 12.5 Hz, 1H, Ar); 8.12 (t, J 8.4 Hz, 1H, Ar).

### Preparation T: (3-Fluoro-4-nitro-phenyl)-acetic acid

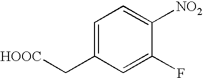

A suspension of (3-fluoro-4-nitro-phenyl)-acetic acid methyl ester (500 mg, 1.0 equiv) in HCl 1M (5 mL, 2.0 equiv) was heated through microwave irradiation for 5 min at 150° C. A solid precipitated while cooling at R.T. and was collected by filtration and dried, to give the product as a white solid in 90% yield.

¹H-NMR (400 MHz, D6-DMSO): 3.78 (s, 2H, CH₂); 7.35 (d, J 8.7 Hz, 1H, Ar); 7.52 (dd, J 1.8 Hz, J 12.5 Hz, 1H, Ar); 8.12 (t, J 8.4 Hz, 1H, Ar).

M/Z (M+H)⁺=no mass ion.

### Preparation U: (2-(3-Fluoro-4-nitro-phenyl)-1-morpholin-4-yl-ethanone

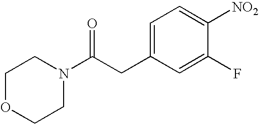

A mixture of (3-fluoro-4-nitro-phenyl)-acetic acid (360 mg, 1.0 equiv), morpholine (475 μL, 1.1 equiv), HATU (756 mg, 3.0 equiv) and pyridine (400 μL, 2.8 equiv) in dimethylformamide (3.6 mL) was stirred at R.T. for 3 days. The reaction mixture was diluted with AcOEt (15 mL) and was washed with water (3*10 mL), brine (10 mL), dried over MgSO₄ and concentrated under reduced pressure. Purification by flash chromatography (MeOH 1% in CH₂Cl₂) gave the product in 51% yield.

¹H-MMR (400 MHz, D6-DMSO): 3.50-3.59 (m, 8H, 4 CH₂); 3.90 (s, 2H, CH₂); 7.29 (d, J 8.6 Hz, 1H, Ar); 7.44 (dd, J 1.8 Hz, J 12.7 Hz, 1H, Ar); 8.10 (t, J 8.4 Hz, 1H, Ar).

M/Z (M+H)⁺=269.

### Preparation V: 4-[2-(3-Fluoro-4-nitro-phenyl)-ethyl]-morpholine

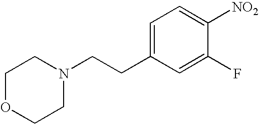

To a 1M solution of BH₃ in THF (2.8 mL, 3.0 equiv), a solution of (2-(3-fluoro-4-nitro-phenyl)-1-morpholin-4-yl-ethanone (250 mg, 1.0 equiv) in anhydrous THF (2.2 mL) was added dropwise. The resulting mixture was stirred at R.T. for 5 Hrs. The reaction mixture was then cooled with an ice bath and hydrolyzed with HCl 2M (1.8 mL, 3.6 equiv). The mixture was concentrated under reduced pressure, HCl 6M (5 mL) was added and the resulting mixture was heated at reflux for 1.25 Hr. It was neutralized with NaOH 5M (6 mL) and extracted with AcOEt (3*15 mL). The organic layer was washed with brine (15 mL), dried over MgSO₄ and concentrated under reduced pressure. Purification by flash chromatography (MeOH 2% in CH₂Cl₂) gave the product as a yellow oil in 33% yield.

¹H-NMR (400 MHz, D6-DMSO): 2.41 (m, 4H, 2 CH₂); 2.56 (t, J 7.7 Hz, 2H, NCH₂); 2.87 (t, J 7.7 Hz, 2H, NCH₂); 3.55 (m, 4H, 2 OCH₂); 7.33 (d, J 8.7 Hz, 1H, Ar); 7.50 (dd, J 1.8 Hz, J 12.8 Hz, 1H, Ar); 8.07 (t, J 8.4 Hz, 1H, Ar).

M/Z (M+H)⁺=255.

### Preparation W: 1-[5-(2-Morpholin-4-yl-ethyl)-2-nitro-phenyl]-4-phenyl-1H-pyrrole-2-carboxylic acid methyl ester, trifluoroacetic acid salt

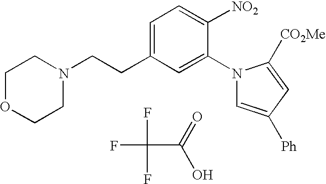

A mixture of 4-[2-(3-fluoro-4-nitro-phenyl)-ethyl]-morpholine (39 mg, 1.2 equiv), 4-phenyl-1H-pyrrole-2-carboxylic acid methyl ester (40 mg, 1.0 equiv) and cesium carbonate (61 mg, 1.2 equiv) in dimethylformamide (1.5 mL) was heated through microwave irradiation for 10 min at 130° C. The reaction mixture was diluted with AcOEt (15 mL) and was washed with water (3*10 mL), brine (10 mL), dried over MgSO₄ and concentrated under reduced pressure. Purification by preparative HPLC gave the product as a TFA salt in 17% yield.

M/Z (M+H)⁺=436.

### Preparation X: 4-(4-Fluoro-3-nitro-phenyl)-morpholine

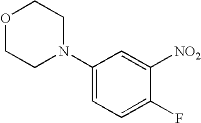

A mixture of 4-fluoro-3-nitro-phenylamine (200 mg, 1.0 equiv), 1-bromo-2-(2-bromo-ethoxy)-ethane (485 μL, 3.0 equiv), potassium carbonate (1.0 g, 6.0 equiv) and potassium iodide (425 mg, 2.0 equiv) in acetonitrile (4 mL) was heated through microwave irradiation for 20 min at 200° C. The reaction mixture was diluted with AcOEt and was washed with water, brine, dried over MgSO₄ and concentrated under reduced pressure. Purification by flash chromatography (10-40% AcOEt in cyclohexane) gave the product in 31% yield.

¹H-NMR (400 MHz, CDCl₃): 3.17-3.20 (m, 4H, 2 NCH₂); 3.88-3.90 (m, 4H, 2 OCH₂); 7.12-7.23 (m, 2H, Ar); 7.51 (m, 1H, Ar).

M/Z (M+H)⁺=227.

### Preparation Y: 1-(4-Morpholin-4-yl-2-nitro-phenyl)-4-phenyl-1H-pyrrole-2-carboxylic acid methyl ester

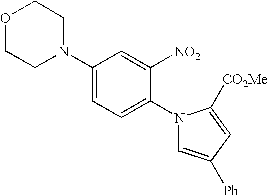

A mixture of 4-(4-fluoro-3-nitro-phenyl)-morpholine (270 mg, 1.0 equiv), 4-phenyl-1H-pyrrole-2-carboxylic acid methyl ester (240 mg, 1.0 equiv) and cesium carbonate (465 mg, 1.2 equiv) in dimethylformamide (8 mL) was heated at 120° C. for 16 Hrs. The reaction mixture was diluted with AcOEt and was washed with water, brine, dried over MgSO₄ and concentrated under reduced pressure. The residue was triturated in AcOEt (5 mL), filtered off, washed with Et₂O and dried under vacuum. The solids were combined to give the product in 70% yield.

M/Z (M+H)⁺=408.

### Preparation Z: 4-[2-(3-Fluoro-4-nitro-phenoxy)-ethyl]-morpholine

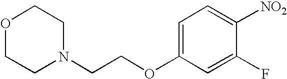

To a solution of triphenylphosphine (835 mg, 1.0 equiv) in anhydrous THF (15 mL) under nitrogen stream, 3-fluoro-4-nitro-phenol (500 mg, 1.1 equiv) and diisopropylazodicarboxyl (625 μL, 1.1 equiv) were added. The resulting mixture was stirred at R.T. for 30 min, and then 2-morpholin-4-yl-ethanol (350 μL, 1.0 equiv) was added. The mixture was stirred at R.T. for 16 Hrs, then was diluted with AcOEt and washed with a saturated solution of NH₄Cl and brine. The organic layer was dried over MgSO₄ and concentrated under reduced pressure. Purification by flash chromatography (1-5% MeOH in CH₂Cl₂) gave the product contaminated with triphenylphosphine oxide.

¹H-NMR (400 MHz, CDCl₃): 2.58 (m, 4H, 2 NCH₂); 2.84 (t, J 5.6 Hz, 2H, NCH₂); 3.74 (m, 4H, 2 OCH₂), 4.19 (t, J 5.6 Hz, 2H, OCH₂); aromatic signals under triphenylphosphine oxide signals.

M/Z (M+H)⁺=271.

### Preparation AA: 1-[5-(2-Morpholin-4-yl-ethoxy)-2-nitro-phenyl]-4-phenyl-1H-pyrrole-2-carboxylic acid methyl ester, chlorhydrate

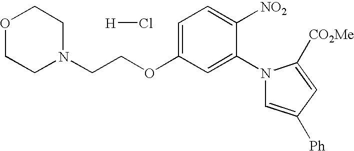

The compound was synthesized according to preparation Y from 4-[2-(3-fluoro-4-nitro-phenoxy)-ethyl]-morpholine (865 mg, 1.0 equiv). The product was purified by flash chromatography (1-2% MeOH in CH₂Cl₂) to give the free base as an oil, which was dissolved in HCl 1M. A solid precipitated, it was collected and dried to afford the corresponding chlorhydrate in 41% yield.

¹H-NMR (400 MHz, D6-DMSO): 3.21 (m, 2H, NCH₂); 3.91 (m, 4H, 2 OCH₂); 4.63 (t, J 5.0 Hz, 2H, OCH₂); 7.23 (t, J 7.5 Hz, 1H, Ar); 7.31-7.40 (m, 4H, Ar); 7.50 (d, J 2.0 Hz, 1H, Ar); 7.69 (dd, J 1.0 Hz, J 8.0 Hz, 2H, Ar); 7.83 (d, J 1.9 Hz, 1H, Ar); 11.49 (bs, 1H, NH⁺); 4 proton signals (2 NCH₂) are missing.

M/Z (M+H)⁺=452.

### Example 1

### 2-Phenyl-5H-pyrrolo[1,2-a]quinoxalin-4-one

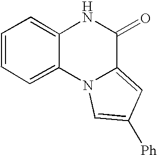

A mixture of compound from preparation B (500 mg, 1 equiv), phenylboronic acid (348 mg, 1.5 equiv), sodium carbonate (604 mg, 3 equiv) and Pd/C (2.5 mg, 0.5%) in methanol/water (4:1)(10 mL) was heated through microwave irradiation 10 min at 150° C. (Pₘₐₓ 70 W). A white solid was obtained, was filtered off and washed with water. The solid was then dissolved in DMF in order to remove the catalyst by filtration. Water was added to the filtrate to re-precipitate a white solid that was filtered off, washed with water, Et₂O and dried to afford the desired product in 80% yield.

Alternatively this compound can be obtained from compound from preparation C following the procedure described in preparation B in 61% yield as a fluffy white solid.

¹H-NMR (400 MHz, D6-DMSO): 7.22-7.34 (m, 4H, Ar); 7.40-7.48 (m, 3H, Ar); 7.82 (d, J 7.4 Hz, 2H, Ar); 8.12 (d, J 7.7, 1H, Ar); 8.72 (s, 1H, Ar); 11.32 (bs, 1H, NH).

M/Z (M+H)⁺=261.

Mp=301-303° C.

### Example 2

### 2-(4-Methoxy-phenyl)-5H-pyrrolo[1,2-a]quinoxalin-4-one

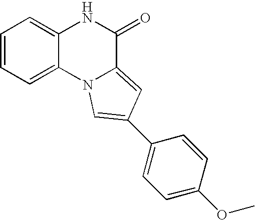

Compound obtained from compound from preparation D in 85% yield as a cream fluffy solid following the procedure described in preparation B.

¹H-NMR (400 MHz, D6-DMSO): 3.79 (s, 3H, OCH₃); 7.00 (d, J 8.7 Hz, 2H, Ar); 7.22-7.32 (m, 3H, Ar); 7.38 (d, J 1.6 Hz, 1H, Ar); 7.75 (d, J 8.8 Hz, 2H, Ar); 8.09 (d, J 7.9 Hz, 1H, Ar); 8.61 (d, J 1.6 Hz, 1H, Ar); 11.29 (bs, 1H, NH).

M/Z (M+H)⁺=291.

Mp=327-332° C.

### Example 3

### Phenyl-(2-phenyl-pyrrolo[1,2-a]quinoxalin-4-yl)-amine

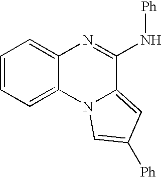

To a solution of compound from preparation E (60 mg, 1 equiv) in DMF (1 mL) was added aniline (39 μL, 2 equiv) and the mixture was heated through microwave irradiation 10 min at 150° C. The reaction mixture was then diluted in AcOEt, washed with water, brine and dried over MgSO₄. The organic layer was concentrated to give a yellow oil. Purification by flash chromatography (5% AcOEt in cyclohexane) afforded the desired product as a white solid in 55% yield.

¹H-NMR (400 MHz, D6-DMSO): 7.05 (t, J 7.3 Hz, 1H, Ar); 7.29-7.41 (m, 5H, Ar); 7.48 (m, 2H, Ar); 7.60-7.63 (m, 1H, Ar); 7.80-7.84 (m, 3H, Ar); 8.10 (dd, J 1.2 Hz, J 8.7 Hz, 2H, Ar); 8.17-8.20 (m, 1H, Ar); 8.86 (d, J 1.7, 1H, Ar); 9.18 (bs, 1H, NH).

M/Z (M+H)⁺=336.

Mp=172° C.

### Example 4

### Ethyl-(2-phenyl-pyrrolo[1,2-a]quinoxalin-4-yl)-amine hydrochloride

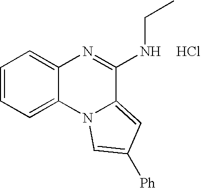

A suspension of compound from preparation E (70 mg, 1 equiv) in ethylamine 2M in THF (1 mL) was heated through microwave irradiation 2×10 min at 150° C., 2×5 min at 180° C. and 10 min at 180° C. The reaction mixture was hydrolysed with HCl 1M and diluted with AcOEt. A white solid appeared and was filtered off, washed with water, AcOEt, Et₂O and dried to give the desired product as the corresponding hydrochloride salt in 37% yield. —H-NMR (400 MHz, D6-DMSO): 1.36 (t, J 7.0 Hz, 3H, NCH₂CH₃) 3.82 (q, J 7.0 Hz, J 7.0 Hz, 2H, CH₃CH₂N); 7.34 (t, J 7.4 Hz, 1H, Ar); 7.16-7.52 (m, 4H, Ar); 7.78 (d, J 7.2 Hz, 2H, Ar); 8.13 (bs, 1H, Ar); 8.29 (m, 2H, Ar); 9.07 (s, 1H, Ar); 10.21 (bs, 1H, NH); 12.56 (bs, 1H, NH).

M/Z (M+H)⁺=288.

Mp=299-301° C.

### Example 5

### Benzyl-(2-phenyl-pyrrolo[1,2-a]quinoxalin-4-yl)-amine

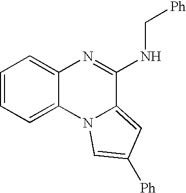

Compound obtained from compound from preparation E in 13% yield as a white solid following the procedure described in example 3.

¹H-NMR (400 MHz, D6-DMSO): 4.80 (d, J 5.9 Hz, 2H, NCH₂Ph); 7.20-7.36 (m, 6H, Ar); 7.41-7.48 (m, 5H, Ar); 7.54 (d, J 1.7 Hz, 1H, Ar); 7.74-7.77 (m, 2H, Ar); 7.99 (t, J 5.7 Hz, J 5.9 Hz, 1H, Ar); 8.10 (dd, J 1.6 Hz, J 7, 9 Hz, 1H, Ar); 8.74 (bd, J 1.6 Hz, 1H, NH).

M/Z (M+H)⁺=350.

### Example 6

### Dimethyl-(2-phenyl-pyrrolo[1,2-a]quinoxalin-4-yl)-amine

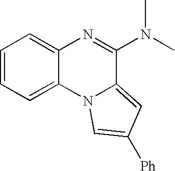

Isolated as a side product (white solid, 48% yield) during the purification of the compound described in example 5.

¹H-NMR (400 MHz, D6-DMSO): 3.38 (s, 6H, (CH₃)₂N); 7.19-7.31 (m, 3H, Ar); 7.41-7.48 (m, 4H, Ar); 7.88 (m, 2H, Ar); 8.11 (dd, J 1.4 Hz, J 8.1 Hz, 1H, Ar); 8.80 (d, J 1.5 Hz, 1H, Ar).

M/Z (M+H)⁺=288.

Mp=109-111° C.

### Example 7

### Phenethyl-(2-phenyl-pyrrolo[1,2-a]quinoxalin-4-yl)-amine

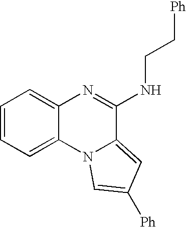

Compound obtained from compound from preparation E in 25% yield as a white solid following the procedure described in example 3.

¹H-NMR (400 MHz, D6-DMSO): 3.02 (t, J 7.8 Hz, 2H, NCH₂CH₂Ph); 3.76 (m, 2H, NCH₂CH₂Ph); 7.19-7.36 (m, 8H, Ar); 7.43-7.58 (m, 5H, Ar); 7.75 (dd, J 1.2 Hz, J 8.3 Hz, 2H, Ar); 8.09 (dd, J 1.4 Hz, J 8.0 Hz, 1H, Ar); 8.72 (bd, J 1.6 Hz, 1H, NH).

M/Z (M+H)⁺=364.

Mp=126° C.

### Example 8

### 2-Phenyl-5H-pyrrolo[1,2-a]quinoxaline-4-thione

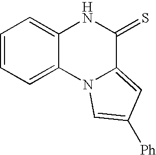

A suspension of compound from example 1 (100 mg, 1 equiv) and Lawesson's reagent (78 mg, 0.5 equiv) in a mixture of toluene (2.5 mL) and acetonitrile (1 mL) was heated through microwave irradiation 10 min at 130° C. A solid appeared and was filtered off, washed with Et₂O and dried to give a pale yellow solid in 76% yield.

¹H-NMR (400 MHz, D6-DMSO): 7.30 (t, J 7.4, 1H, Ar); 7.43 (m, 4H, Ar); 7.59 (m, 1H, Ar); 7.68 (d, J 1.7 Hz, 1H, Ar); 7.86 (dd, J 1.2 Hz, J 8.3 Hz, 2H, Ar); 8.21 (m, 1H, Ar); 8.90 (d, J 1.7 Hz, 1H, Ar); 12.99 (bs, 1H, NH).

M/Z (M+H)⁺=277.

### Example 9

### 4-Methylsulfanyl-2-phenyl-pyrrolo[1,2-a]quinoxaline

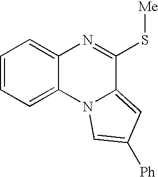

A solution of compound from example 8 (60 mg, 1 equiv) in THF (2 mL) was purged with N₂ and cooled to 0° C., then DBU (47 μL, 1.2 equiv) and methyl iodide (16 μL, 1.2 equiv) were added. The mixture was stirred at 0° C. under N₂ for 1 hour, then the reaction mixture was diluted with AcOEt and washed with water, brine and dried over MgSO₄. The organic layer was concentrated to give a pale yellow solid. After trituration in a mixture of pentane and Et₂O a cream solid was isolated and dried to afford the desired product in 60% yield.

¹H-NMR (400 MHz, D6-DMSO): 2.73 (s, 3H, CH₃—S); 7.30 (t, J 7.6 Hz, 1H, Ar); 7.33 (d, J 1.5 Hz, 1H, Ar); 7.42-7.50 (m, 3H, Ar); 7.56 (t, J 7.7 Hz, 1H, Ar); 7.82 (dd, J 1.3 Hz, J 7.3 Hz, 1H, Ar); 7.88-7.90 (m, 2H, Ar); 8.30 (dd, J 1.2 Hz, J 8.2 Hz, 1H, Ar); 9.97 (d, J 1.51 Hz, 1H, Ar).

M/Z (M+H)⁺=291.

Mp=132° C.

### Example 10

### 2-(4-Dimethylamino-phenyl)-5H-pyrrolo[1,2-a]quinoxalin-4-one, trifluoroacetic acid salt

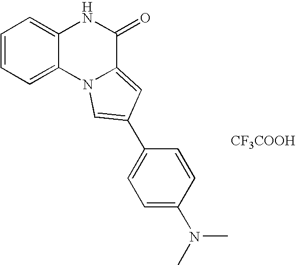

A suspension of compound from preparation L (120 mg, 1 equiv) and iron powder (440 mg, 2.2 equiv) in acetic acid (1 mL) was heated through microwave irradiation 10 min at 130° C. The reaction mixture was filtered off, and the filtrate was diluted with AcOEt. The organic layer was extracted with HCl 1M. The aqueous layer was basified with NaOH 6N and a solid precipitated. Purification of the solid by flash chromatography (20% to 100% AcOEt in cyclohexane) followed by purification by preparative chromatography afforded the product as a TFA salt.

M/Z (M+H)⁺=304.

### Example 11

### 8-Morpholin-4-yl-2-phenyl-5H-pyrrolo[1,2-a]quinoxalin-4-one, trifluoroacetic acid salt

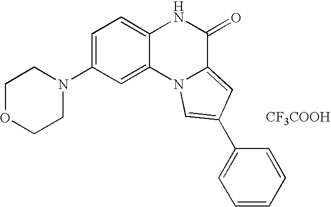

A suspension of compound from preparation J (110 mg, 1 equiv) and palladium 10% on carbon (10 mg) in a mixture of acetic acid (0.5 mL) and ethanol (5 mL) was hydrogenated overnight at R.T. under hydrogen atmosphere. The suspension was filtered off and washed with ethanol. The solid was then dissolved in hot DMF and filtered off. Water and NaHCO₃ were added to the filtrate, and the mixture was extracted with AcOEt. The organic layer was concentrated and was purified by preparative chromatography to give the product as a TFA salt.

M/Z (M+H)⁺=346.

### Example 12

### 8-Methyl-2-phenyl-5H-pyrrolo[1,2-a]quinoxalin-4-one

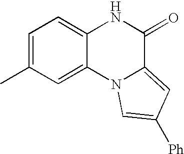

A suspension of 1-(5-methyl-2-nitro-phenyl)-4-phenyl-1H-pyrrole-2-carboxylic acid methyl ester (50 mg, 1.0 equiv) and iron powder (33 mg, 4.0 equiv) in acetic acid (2 mL) was heated through microwave irradiation for 8 min at 150° C. A solid precipitated. The reaction mixture was hydrolyzed with HCl 1M (10 mL) and extracted with AcOEt (10 mL). The organic layer was washed with water (3*10 mL), dried over MgSO₄ and concentrated under reduced pressure. The residue was triturated in Et₂O, filtered off and dried under vacuum to give the product in 17% yield.

¹H-NMR (400 MHz, D6-DMSO): 2.41 (s, 3H, CH₃); 7.12 (dd, J 1.2 Hz, J 8.2 Hz, 1H, Ar); 7.20 (d, J 8.1 Hz, 1H, Ar); 7.27 (t, J 7.3 Hz, 1H, Ar); 7.40-7.44 (m, 3H, Ar); 7.81 (dd, J 1.2 Hz, J 8.3 Hz, 2H, Ar); 7.97 (s, 1H, Ar); 8.69 (d, J 1.7 Hz, 1H, Ar); 11.21 (s, 1H, NH).

M/Z (M+H)⁺=276.

Mp: 314-315° C.

### Example 13

### 8-Chloro-2-phenyl-5H-pyrrolo[1,2-a]quinoxalin-4-one

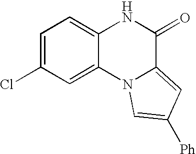

A suspension of 1-(5-chloro-2-nitro-phenyl)-4-phenyl-1H-pyrrole-2-carboxylic acid methyl ester (50 mg, 1.0 equiv) and iron powder (31 mg, 4.0 equiv) in acetic acid (2 mL) was heated through microwave irradiation for 8 min at 150° C. A solid precipitated. The reaction mixture was hydrolyzed with HCl 1M. The remaining solid was filtered off, washed with HCl 1M, MeOH, Et₂O and dried under vacuum to give the product as a yellow solid in 55% yield.

¹H-NMR (400 MHz, D6-DMSO): 7.30 (m, 2H, Ar); 7.36 (m, 1H, Ar); 7.43 (m, 2H, Ar); 7.48 (d, J 1.5 Hz, 1H, Ar); 7.81 (dd, J 1.1 Hz, J 8.3 Hz, 2H, Ar); 8.30 (d, J 2.1 Hz, 1H, Ar); 8.79 (d, J 1.7 Hz, 1H, Ar); 11.40 (s, 1H, NH).

M/Z (M+H)⁺=295.

Mp: 346° C.

### Example 14

### 8-(4-Methyl-piperazin-1-yl)-2-phenyl-5H-pyrrolo[1,2-a]quinoxalin-4-one

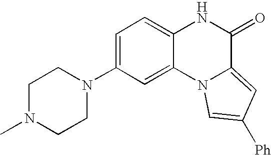

To a solution of 1-[5-(4-methyl-piperazin-1-yl)-2-nitro-phenyl]-4-phenyl-1H-pyrrole-2-carboxylic acid methyl ester (190 mg, 1.0 equiv) in acetic acid (5 mL) was added palladium 10% on charcoal (40 mg). The mixture was purged with hydrogen and a pressure of hydrogen was maintained for 3 days. The catalyst was filtered off on celite and the filtrate was concentrated. Purification by flash chromatography (NH₃/MeOH/CH₂Cl₂ (1:10:90)) afforded the product as a yellow solid in 74% yield.

¹H-NMR (400 MHz, D6-DMSO): 2.25 (s, 3H, NCH₃); 3.23 (m, 4H, 2 NCH₂); 6.95 (dd, J 2.6 Hz, J 9.2 Hz, 1H, Ar); 7.16 (d, J 8.8 Hz, 1H, Ar); 7.27 (m, 1H, Ar); 7.41 (m, 3H, Ar); 7.59 (d, J 2.3 Hz, 1H, Ar); 7.83 (dd, J 1.1 Hz, J 8.2 Hz, 2H, Ar); 8.78 (d, J 1.8 Hz, 1H, Ar); 11.08 (s, 1H, NH).

4 proton signals (2 OCH₂) are missing.

M/Z (M+H)⁺=359.

### Example 15

### 8-(3-Morpholin-4-yl-propylamino)-2-phenyl-5H-pyrrolo[1,2-a]quinoxalin-4-one, trifluoro-acetic acid salt

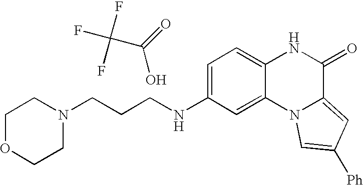

To a solution of 1-[5-(3-morpholin-4-yl-propylamino)-2-nitro-phenyl]-4-phenyl-1H-pyrrole-2-carboxylic acid methyl ester (110 mg, 1.0 equiv) in acetic acid (4 mL) was added palladium 10% on charcoal (30 mg). The mixture was purged with hydrogen and a pressure of hydrogen was maintained for 3 days. The catalyst was filtered off on celite. The filtrate was diluted with water (20 mL), neutralized with NaHCO₃ and extracted with AcOEt (3*20 mL). The organic layer was washed with brine (20 mL), dried over MgSO₄ and concentrated under reduced pressure. Purification by preparative HPLC afforded the product as a brown solid in 15% yield.

¹H-NMR (400 MHz, D6-DMSO): 1.98 (m, 2H, CH₂); 3.10 (m, 2H, CH₂); 3.24 (m, 6H, 3 CH₂); 3.65 (m, 2H, CH₂); 3.97 (m, 2H, CH₂); 6.64 (dd, J 2.2 Hz, J 8.7 Hz, 1H, Ar); 7.09 (d, J 8.7 Hz, 1H, Ar); 7.17 (d, J 2.3 Hz, 1H, Ar); 7.27 (m, 1H, Ar); 7.42 (m, 3H, Ar); 7.82 (dd, J 1.2 Hz, J 8.3 Hz, 2H, Ar); 8.61 (d, J 1.7 Hz, 1H, Ar); 11.00 (s, 1H, NH).

M/Z (M+H)⁺=403.

### Example 16

### 7-Morpholin-4-yl-2-phenyl-5H-pyrrolo[1,2-a]quinoxalin-4-one, chlorhydrate

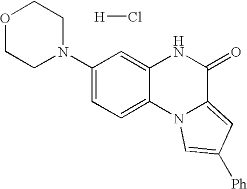

A suspension of 1-(4-morpholin-4-yl-2-nitro-phenyl)-4-phenyl-1H-pyrrole-2-carboxylic acid methyl ester (50 mg, 1.0 equiv) and iron powder (40 mg, 6.0 equiv) in acetic acid (1 mL) was heated through microwave irradiation for 2*10 min at 130° C. The reaction mixture was filtered and the filtrate was concentrated under reduced pressure. The residue was triturated in HCl 1M and a solid was collected by filtration. It was then dissolved in a 1.25 M solution of HCl in MeOH and stirred at R.T. for 16 Hrs. A solid precipitated, it was filtered off and dried to give the chlorhydrate as a white solid in 60% yield.

¹H-NMR (400 MHz, D6-DMSO): 3.15 (m, 4H, 2 NCH₂); 3.78 (m, 4H, 2 OCH₂); 6.84 (bs, 1H, Ar); 6.96 (m, J 7.9 Hz, 1H, Ar); 7.25 (t, J 7.3 Hz, 1H, Ar); 7.41 (t, J 8.0 Hz, 2H, Ar); 7.79 (m, J 7.5 Hz, 2H, Ar); 7.99 (m, J 8.9 Hz, 1H, Ar); 8.61 (s, 1H, Ar); 11.15 (s, 1H, NH). M/Z (M+H)⁺=246.

### Example 17

### 8-(2-Morpholin-4-yl-ethoxy)-2-phenyl-5H-pyrrolo[1,2-a]quinoxalin-4-one, trifluoroacetic acid salt

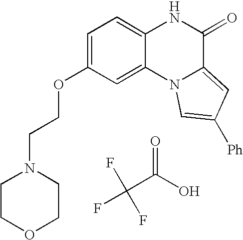

To a solution of 1-[5-(2-morpholin-4-yl-ethoxy)-2-nitro-phenyl]-4-phenyl-1H-pyrrole-2-carboxylic acid methyl ester chlorhydrate (1.1 g, 1.0 equiv) in acetic acid (22 mL) was added palladium 10% on charcoal (100 mg). The mixture was purged with hydrogen and a pressure of hydrogen was maintained for 5 days. The reaction mixture was hydrolyzed with HCl 1M (50 mL) and the catalyst was filtered off on celite. The filtrate was washed with AcOEt and Et₂O. The aqueous layer was made basic by addition of NaOH pellets and was extracted with AcOEt. The organic layer was washed with brine, dried over MgSO₄ and concentrated under reduced pressure. Purification by flash chromatography (MeOH 1-10% in CH₂Cl₂) followed by purification by preparative HPLC gave the product as a trifluoroacetic acid salt in less than 10% yield.

¹H-NMR (400 MHz, D6-DMSO): 3.26 (m, 2H, NCH₂); 3.63 (m, 2H, OCH₂); 4.00 (m, 2H, OCH₂); 4.47 (m, 2H, OCH₂); 7.00 (m, 1H, Ar); 7.25-7.30 (m, 2H, Ar); 7.41-7.45 (m, 3H, Ar); 7.77-7.81 (m, 3H, Ar); 8.72 (d, J 1.6 Hz, 1H, Ar); 10.25 (bs, 1H, NH⁺); 11.23 (bs, 1H, NH); 4 proton signals (2 NCH₂) are missing.

M/Z (M+H)⁺=390.

Mp: 190-200° C.

### Example 18

### Evaluation of the Pharmacological Activities of the Compounds

### Human A₁ Receptor:

Compounds of the invention were evaluated in binding assays following procedure described by Townsend-Nicholson and Schofield (J. Biol. Chem. (1994), 269:2373-2376). Human A₁ receptors were used in CHO cells with [³H]DPCPX (1 nM) as radioligand, DPCPX (1 μM) for non-specific binding and 60 minutes of incubation at 22° C.

### Human A_{2A} Receptor:

Compounds of the invention were evaluated in binding assays following procedure described by Luthin et al. (Mol. Pharmacol. (1995), 47:307-313). Human A_{2A} receptors were used in HEK-293 cells with [³H]CGS 21680 (6 nM) as radioligand, NECA (10 μM) for non-specific binding and 90 minutes of incubation at 22° C.

### Human A_{2B} Receptor:

Compounds of the invention were evaluated in binding assays following procedure described by Stehle et al. (Mol. Endocrinol. (1992), 6:384-393). Human A_{2B} receptors were used in HEK-293 cells with [³H]MRS1754 (0.5 nM) as radioligand, NECA (1 μM) for non-specific binding and 120 minutes of incubation at 22° C.

### Human A₃ Receptor:

Compounds of the invention were evaluated in binding assays following procedure described by Salvatore et al. (Proc. Natl. Acad. Sci. (1993), 90:10365-10369). Human A₃ receptors were used in HEK-293 cells with [¹²⁵I]AB-MECA (0.15 nM) as radioligand, IB-MECA (1 μM) for non-specific binding and 90 minutes of incubation at 22° C. FIG. 1 shows competition curve obtained with compound of example 1 at the human A₃ receptor (hA₃).

The results for the compound of example 1 are summarized in the following table (I):

TABLE (I)

| | hA₁ IC₅₀ | hA_{2A} IC₅₀ | hA_{2B} IC₅₀ | hA₃ IC₅₀ |
|---|---|---|---|---|
| Example 1 | >10 μM | >10 μM | >1 μM | 9.3 nM |
| Example 2 | ND | ND | ND | 82% inhibition |
| | | | | at 1 μM |
| Example 10 | ND | ND | ND | 32% inhibition |
| | | | | at 1 μM |
| Example 11 | 53% | >10 μM | >10 μM | 90% inhibition |
| | inhibition | | | at 1 μM |
| | at 10 μM | | | |
| Example 14 | 8.4 μM | >10 μM | >10 μM | 11 nM |
| Example 15 | 53% | 69% | >10 μM | 3.1 nM |
| | inhibition | inhibition | | |
| | at 10 μM | at 10 μM | | |
| Example 16 | ND | ND | ND | / |
| Example 17 | 54% | 57% | >10 μM | 3.5 nM |
| | inhibition | inhibition | | |
| | at 10 μM | at 10 μM | | |

ND: not done

### Functional Assay on Human A₃ Receptor:

CHO cells expressing the human A₃ receptor were grown overnight as a monolayer in 24 well tissue culture plates (400 μl/well; 2×105 cells/well). cAMP generation was performed in Dulbecco's modified Eagle's medium (DMEM)/N-2-hydroxyethylpiperazin-N-2-ethansulfonic acid (HEPES) buffer (0.60 g HEPES/50 ml DMEM pH 7.4). Each well was washed twice with HEPES/DMEM buffer (250 μl), and the following added: rolipram (50 μM) and cilostamide (50 μM). This was incubated for 30 min at 37° C. followed by the introduction of the compound of example 1 (10 μM) and Cl-IB-MECA (0.1 μM) or DMEM/HEPES. After a further 10 min of incubation, forskolin was added (10 μM). After a subsequent 15 min, incubation was stopped by aspirating the assay medium and by adding 200 μl of ice-cold 0.1M HCl. The amount of cAMP was determined by competition with [3H]cAMP for protein kinase A (PKA). Briefly, the sample, approximately 1.8 nM [3H]cAMP, and 100 μl of PKA solution were incubated on ice for at least 2.5 h. The incubations were stopped by rapid dilution with 2 ml of icecold Tris HCl buffer (pH 7.4), and bound radioactive material was then recovered by filtration through Whatman GF/C filters. Filters were additionally rinsed with 2×2 ml of Tris HCl buffer, and then the radioactivity was counted in Packard Emulsifier Safe scintillation fluid (3.5 mL). All data reflect three independent experiments.

Effects of compound of example 1 on cAMP levels in CHO cells expressing the human A₃ receptor are summarized in the following table (II):

TABLE (II)

| | cAMP levels | | |
|---|---|---|---|
| | n = 1 | n = 2 | n = 3 |
| Basal | 0.000 | 0.000 | 0.000 |
| Forskolin | 100.000 | 100.000 | 100.000 |
| Example 1 | 150.133 | 130.150 | 104.510 |
| 2-Cl-IB-MECA | 23.252 | 23.252 | 14.006 |
| 2-Cl-IB-MECA + | 77.540 | 59.992 | 54.466 |
| example 1 | | | |

### Discussion:

Results show that compound of example 1 is a highly potent ligand for A₃ receptor. In addition, the compound of example 1 is selective for the subtype 3 for the adenosine receptor family as no detectable affinity was measured on A₁ nor A_{2A} up to 10 μM and on A_{2B} up to 1 μM. Compound of example 1 acts as an antagonist for A₃ receptor as it is able to prevent cAMP-decrease evoked by the reference agonist Cl-IB-MECA.

Compound of example 2 also shows potent affinity on A₃ receptor as it inhibits 82% of the specific binding at the concentration of 1 μM.

### Conclusion:

Compound of example 1 is a potent and selective antagonist for the A₃ receptor.

### Example 19

### In Vitro Evaluation of the Cytotoxic Activity

This study aims to determine the cytotoxic activity of a compound of the invention against a panel of ten human cancer cell lines.

### Test:

Paclitaxel (Taxol®, ref. T1912, Sigma) is used as positive control.

Human tumor cells were plated at 5,000 to 10,000 cells per well, with 24 h incubation time before treatment. Tumor cell lines were incubated for 96 hours with 10 concentrations, in ¼ dilution steps, of compound of the invention (from 3.8×10⁻¹⁰ to 1.0×10⁻⁴ M) and Paclitaxel (from 3.8×10⁻¹³ to 1.0×10⁻⁷ M). Experiments were repeated three times, each concentration being issued from quadruplicate. Control cells were treated with corresponding vehicle alone. At the end of the treatments, the cytotoxic activity was evaluated by a MTS assay (Baltrop J A et al, Bioorg. Med. Chem. Lett. 1991, 611-4).

### Results:

Means and standard deviations (SD) of tested compound IC₅₀ determination experiments on a panel of human cancer cells.

| | | |
|---|---|---|
| Human cell | Paclitaxel (nM) | Tested compound (μM) |

| lines | Mean | SD | Mean | SD |
|---|---|---|---|---|
| A2780 | 76.95 | 2.73 | 6.49 | 1.24 |
| A375 | 6.52 | N.A. | 10.78 | 2.31 |
| HCT 116 | 5.57 | 3.35 | 8.25 | 3.86 |
| HL-60 | 6.65 | 2.05 | 9.02 | 3.43 |
| HT-29 | 8.29 | 6.42 | 13.21 | 7.54 |
| K-562 | 7.60 | 3.24 | 9.85 | 5.03 |
| PANC-1 | >100 | — | 15.26 | 3.15 |
| PC-3 | 8.80 | 3.18 | 11.65 | 7.07 |
| U-87 MG | >100 | — | 11.63 | 2.50 |
| U-937 | 5.29 | 1.56 | 7.70 | 2.53 |

### Conclusion:

The compound of the invention shows an antiproliferative activity against the panel of ten human cancer cell lines tested with IC₅₀ values in the ten micromolar range.

1. A compound of general formula (I): 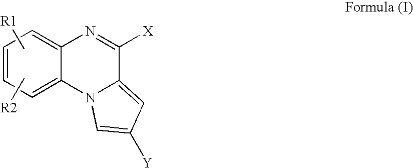
wherein:
R1 and R2 are independently from each other hydrogen, halogen, CN, CF₃, OCF₃, alkyl, COOH, O-(alkyl), S-(alkyl), N-(lower alkyl)(alkyl), heterocycloalkyl, aryl or heteroaryl, NH-(alkyl);
X represents R3 or COR3, O—R3 or S—R3, N—R3R4, NHCOR3, N(lower alkyl)COR3, NHSO₂R3, N(lower alkyl) SO₂R3 or NHCONHR3 with
R3 and R4 are independently from the other hydrogen, alkyl, CF₃, aryl, heteroaryl, alkylaryl or alkylheteroaryl;
Y represents a (A)ₙ-B group wherein
n represents either 0 or 1,
A is a moiety selected in the group consisting of O, S, NH, N-(lower alkyl), N-aryl, N-heteroaryl,
B is an aryl or a heteroaryl group;
as well as pharmaceutically acceptable salt thereof, a prodrug of the compound or the salt, or a solvate or hydrate of the compound, the salt or the prodrug.

2. A compound according to claim 1, wherein R1, R2 and x are as defined in claim 1 and Y represents an aryl group.

3. A compound according to claims 1 or 2, wherein R1 is hydrogen, R2 and X is as defined in claim 1 and Y represents an aryl group.

4. A compound according to claim 1 or 2, wherein R1 and R2 are hydrogen, X is as defined in claim 1 and Y represents an aryl group.

5. A compound according to claim 1, wherein R1 and R2 are hydrogen, X represents R3, O—R3 or S—R3, N—R3R4, wherein R3 and R4 are independently from the other hydrogen, alkyl, CF₃, aryl, heteroaryl, alkylaryl or alkylheteroaryl;
and Y represents an aryl group.

6. A compound according to claim 1 selected from 2-Phenyl-5H-pyrrolo[1,2-a]quinoxalin-4-one and 2-(4-Methoxy-phenyl)-5H-pyrrolo[1,2-a]quinoxalin-4-one.

7. A pharmaceutical composition comprising a pharmaceutically effective amount of a compound of formula (I) according to claim 1, or pharmaceutically acceptable salts thereof, in combination with a pharmaceutically acceptable carrier, diluent or excipient.

8. A method of modulating an A₃ receptor functioning by the administration of a therapeutically effective amount of a compound of general formula (I) according to claim 1 to a patient in need thereof, such that modulation of the adenosine receptor's activity occurs.

9. A method of selectively blocking an A₃ receptor of comprising administering a therapeutically effective amount of a compound of formula (I) to a patient in need thereof, such that blockage of the adenosine receptor's activity occurs.

10. A method of treating a patient suffering from or at risk for acquiring an A₃ receptor mediated disorder selected from asthma, hypersensitivity, rhinitis, hay fever, serum sickness, allergic vasculitis, atopic dermatitis, dermatitis, psoriasis, eczema, idiopathic pulmonary fibrosis; eosinophilic chlorecystitis, chronic airway inflammation, chronic obstructive pulmonary disease, hypereosinophilic syndromes, eosinophilic gastroenteritis, edema, urticaria, eosinophilic myocardial disease, episodic angioedema with eosinophilia, inflammatory bowel disease, ulcerative colitis, allergic granulomatosis, carcinomatosis, eosinophilic granuloma, familial histiocytosis, hypertension, mast cell degranulation, tumor, cardiac hypoxia, cerebral ischemia, diuresis, renal failure, neurological disorder, mental disorder, cognitive disorder, myocardial ischemia, bronchoconstriction, arthritis, autoimmune disease, Crohn's disease, Grave's disease, diabetes, multiple sclerosis, anaemia, psoriasis, fertility disorders, lupus erthyematosus, reperfusion injury, brain arteriole diameter, the release of allergic mediators, scleroderma, stroke, global ischemia, central nervous system disorder, cardiovascular disorder, renal disorder, inflammatory disorder, gastrointestinal disorder, eye disorder, allergic disorder, respiratory disorder, or immunological disorder, comprising administering to the patient an amount of a compound of formula (I) effective to modulate A₃ activity in the patient.
